(19) United States
(12) Patent Application Publication
(10) Pub. No.: US 2024/0057617 A1
(43) Pub. Date: Feb. 22, 2024

(54) COMPOSITIONS AND METHODS FOR MEALYBUG MONITORING AND CONTROL

(71) Applicant: The State of Israel, Ministry of Agriculture & Rural Development, Agricultural Research Organization, Rishon-LeZion (IL)

(72) Inventors: Anat ZADA BYERS, Beer-Yaacov (IL); Roy KASPI, Hod-HaSharon (IL); Sara STEINER, Petach-Tikva (IL); Daniela FEFER, Nes-Ziona (IL)

(73) Assignee: The State of Israel, Ministry of Agriculture & Rural Development, Agricultural Research Organization, Rishon-LeZion (IL)

(21) Appl. No.: 17/766,272

(22) PCT Filed: Oct. 4, 2019
(86) PCT No.: PCT/IL2019/051086

§ 371 Date: Apr. 4, 2022

(51) Int. Cl. A01N 65/22; A01M 1/02; A01P 7/04

(52) U.S. Cl. CPC A01N 65/22; A01M 1/026; A01P 7/04

(57) ABSTRACT

A preparation comprising gamma-necrodyl isobutyrate and an agriculturally acceptable carrier is disclosed for the monitoring and control of mealybugs. Methods of synthesizing the preparation and other mealybug pheromones that feature a necrodane structure are also disclosed.

### FIELD AND BACKGROUND OF THE INVENTION

The present invention, in some embodiments thereof, relates to processes of preparing synthetic pheromones usable for the monitoring and control of mealybugs and to the use of a synthetic pheromone for the monitoring and control of mealybugs and, more particularly, mealybugs of the Nipaecoccus viridis species.

The spherical mealybug Nipaecoccus viridis (Newstead) (Hemiptera: Pseudococcidae), is a polyphagous pest that is widespread throughout the tropics and subtropics, attacking numerous plant species. Major crops infested by N. viridis include soybean, citrus, mango, tamarind, pomegranate and grapevines. The mealybug spread from the Indian sub-continent to other parts of the world (Sharaf and Meyerdirk 1987) and often causes considerable damage mostly in citrus orchards of southern and eastern Mediterranean, Australia, Florida, and warm areas of Asia (Griffiths and Derksen 2010). As a major citrus pest, it attacks many citrus varieties and can be found on all parts of the tree: stems, leaves and fruits. High populations of the mealybug on citrus trees cause damage such as fruit dropping, growth distortions, and sometimes even degeneration of the tree. However, most of the damage is due to larvae feeding on fruit. A green mark occurs on ripe fruit at the point where the mealybugs pierce with their mouthparts. This mark disqualifies the fruit for marketing and causes significant economic loses.

It has previously been shown that the winged short-lived males of N. viridis mealybug were attracted to airborne volatile collections (aerations) of the virgin wingless females (Mendel et al. 2012).

There are about 2000 mealybug species in 270 genera (family Pseudococcidae) (Gerson and Applebaum 2015). To date, only 21 of these mealybug species pheromones have been identified—compared to hundreds of pheromones identified in moth pest species (El-Sayed 2019). One reason for this difference is that virgin females of mealybug species release smaller amounts of pheromone than moths (well known to produce in the order of nanograms), which requires separating the sexes of thousands of mealybugs prior to volatile collection. Another reason is that it remains unclear whether mealybugs possess a gland like moths from which pheromone can be extracted. In addition, female mealybugs require feeding on a host plant to release volatiles, while female moths do not. The volatiles of these host plants generally mask the mealybugs' pheromone even when thousands of virgin females are used. Moreover, mealybug pheromones typically exhibit unique structures and thus chemical standards or GC retention indexes are rarely available. All these make the isolation of a mealybug pheromone using the “classical” methods of solvent extraction or adsorbent extraction much more complicated.

Trans-α-necrodol and β-necrodol have been identified in the defensive secretion produced in the rectal gland of the red-lined carrion beetle Necrodes surinamensis (Eisner and Meinwald 1982). Trans-α-Necrodyl isobutyrate was found as the pheromone of females of grape mealybug Pseudococcus maritimus (Figadere et al. 2007). Lately, a necrodane with an unusual β-necrodol skeleton (4,5,5-trimethyl-3-methylenecyclopent-1-en-1-yl)methyl acetate, was identified in citrus mealybug females of Delottococcus aberiae (De Lotto) (Hemiptera: Pseudococcidae) (Vacas et al. 2019).

To date, only twenty one pheromones of mealybugs have been identified as esters of terpene alcohols and carboxylic acids (Unelius et al. 2010; Tabata and Ichiki 2017). The majority of these pheromones have unique structures and therefore are challenging to identify and synthesize. Thus, using mealybug pheromones for monitoring their populations is still rather limited in scope compared with extensive monitoring with baits of moth and beetle pests.

Synthetic processes of preparing mealybug pheromones featuring a necrodane structures are described in Zou et al., J. Agric. Food Chem. 2010, 58, 4977-4982 and Vacas et al., J. Agric. Food Chem. 2019, 67, 9441-9449.

Additional background art includes Pamingle et al. Helv Chim Acta 74: 543-548; and Kashima and Miyazawa, Chemistry & Biodiversity, Vol. 11, page 396 (2014).

### SUMMARY OF THE INVENTION

According to an aspect of some embodiments of the present invention there is provided a preparation comprising γ-necrodyl isobutyrate and, optionally, an agriculturally acceptable carrier.

According to some of any of the embodiments described herein, the γ-necrodyl isobutyrate is synthesized from an essential oil of Lavandula luisieri.

According to some of any of the embodiments described herein, the preparation comprises an antioxidant, and/or a preservative.

According to an aspect of some embodiments of the present invention there is provided a pest control device comprising γ-necrodyl isobutyrate.

According to an aspect of some embodiments of the present invention there is provided a method of controlling a population of Nipaecoccus viridis mealybug population comprising exposing the population to an effective amount of γ-necrodyl isobutyrate, thereby controlling the population of Nipaecoccus viridis.

According to some of any of the embodiments described herein, the exposing is effected by releasing the γ-necrodyl isobutyrate in a location which is frequented by the Nipaecoccus viridis.

According to some of any of the embodiments described herein, the location is a field, vineyard or orchard.

According to some of any of the embodiments described herein, the γ-necrodyl isobutyrate is comprised in a trap.

According to some of any of the embodiments described herein, the γ-necrodyl isobutyrate is comprised in a sustained release preparation.

According to some of any of the embodiments described herein, the orchard comprises trees of a species selected from the group consisting of citrus, mango, tamarind and pomegranate.

According to an aspect of some embodiments of the present invention there is provided a method of monitoring an amount of Nipaecoccus viridis mealybugs present in a location which is frequented by Nipaecoccus viridis: (a) setting a trap comprising γ-necrodyl isobutyrate in the location; and (b) determining the amount of mealybugs in the trap, thereby monitoring the amount of Nipaecoccus viridis mealybugs.

According to some of any of the embodiments described herein, the determining comprises counting the number of Nipaecoccus viridis mealybugs in the trap.

According to an aspect of some embodiments of the present invention there is provided a method of synthesizing γ-necrodol, the method comprising subjecting an α-necrodol to conditions that effect rearrangement of the α-necrodol to thereby generate γ-necrodol.

According to some of any of the embodiments described herein, the α-necrodol is extracted from an essential oil of Lavandula luisieri.

According to some of any of the embodiments described herein, the α-necrodol is trans-α-necrodol.

According to an aspect of some embodiments of the present invention there is provided a method of synthesizing γ-necrodyl isobutyrate, the method comprising: (a) synthesizing γ-necrodol according to the method described herein in any of the respective embodiments; and (b) reacting the γ-necrodol with isobutyryl chloride, isobutyric acid and/or isobutyric anhydride under conditions that produce γ-necrodyl isobutyrate, thereby synthesizing the γ-necrodyl isobutyrate.

According to some of any of the embodiments described herein, the method further comprises purifying the γ-necrodyl isobutyrate following the reacting.

According to an aspect of some embodiments of the present invention there is provided a method of synthesizing trans-α-necrodol isobutyrate, the method comprising: (a) producing trans-α-necrodol from an essential oil of Lavandula luisieri; and (b) reacting the trans-α-necrodol with isobutyric acid, isobutyryl halide and/or isobutyryl anhydride, under conditions that produce trans-α-necrodol isobutyrate, thereby synthesizing the trans-α-necrodol isobutyrate.

According to an aspect of some embodiments of the present invention there is provided a method of synthesizing a mealybug pheromone featuring a necrodane skeleton, the method comprising: producing a necrodol compound from an essential oil of a plant that comprises at least 10% of the necrodol compound and/or an ester thereof; and subjecting the necrodol compound to conditions that effect rearrangement and/or esterification of the necrodol, thereby synthesizing the mealybug pheromone.

According to some of any of the embodiments described herein, the plant is Lavandula luisieri and the necrodol compound is an α-necrodol.

According to some of any of the embodiments described herein, the mealybug pheromone is an ester of α-necrodol, and wherein the compound is synthesized by subjecting the trans-α-necrodol to conditions that effect esterification of the α-necrodol.

According to some of any of the embodiments described herein, the mealybug pheromone is an ester of the necrodol, and is synthesized by subjecting the necrodol to conditions that effect esterification of the necrodol.

According to some of any of the embodiments described herein, the necrodol compound is an α-necrodol and/or a β-necrodol, and wherein the mealybug pheromone is a γ-necrodol or an ester thereof, the method comprising subjecting the α-necrodol and/or a β-necrodol to conditions that effect rearrangement of the necrodol compound, to thereby obtain the γ-necrodol, and optionally further subjecting the γ-necrodol to conditions that effect esterification, to thereby obtain the ester of γ-necrodol.

According to other aspects of some embodiments of the present invention there are provided devices containing and methods utilizing the mealybug pheromone prepared by the method as described herein, as described herein in any of the respective embodiments and any combination thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B show the pheromone release by 300 N. viridis virgin females on potato sprouts as determined by sequential SPME/GC-MS analysis in 9-11 day old females during four sampling periods in sequence (FIG. 1A) and during six days of samplings after the last molt (FIG. 1B) (numbers above daily maximum peak represent the age of the females on the same day).

FIGS. 2A-B present the mass spectrum (MS) of γ-necrodol (FIG. 2A) and γ-necrodyl isobutyrate (FIG. 2B) from N. viridis female aerations.

FIG. 3 presents the 2D chemical structures of γ-necrodol and γ-necrodyl isobutyrate.

FIG. 4 is a bar graph showing the mean numbers of arrested males in Petri dish choice tests. I=γ-necrodol (10 ng), II=γ-necrodyl isobutyrate (10 ng), Mix=I+II (5 ng each), control=n-hexane. Bars with the same letters were not significantly different (N=6).

FIG. 5 is a bar graph showing the mean numbers of flying males trapped in sticky traps in rearing room. I=γ-necrodol (20 μg), II=γ-necrodyl isobutyrate (20 μg), Mix=I+II (20 μg each), control=n-hexane. Bars with the same letters were not significantly different (N=12).

FIG. 6 presents an exemplary synthetic route of γ-necrodol and γ-necrodyl isobutyrate from Lavendula luisieri (Rozeira) essential oil, according to some embodiments of the present invention.

FIG. 7 presents the mass spectra of γ-Necrodol synthetically prepared by Lavendula luisieri (Rozeira) essential oil rearrangement.

FIGS. 8A-B present high resolution mass spectra (HR-MS) (70 eV, 7890B GC/7250 Q-TOF MS, Agilent) of γ-necrodol as obtained from airborne collections from female Nipaecoccus viridis (FIG. 8A), and by Lavendula luisieri (Rozeira) essential oil rearrangement (FIG. 8B).

FIGS. 9A-B present the HR-Mass spectra (70 eV, 7890B GC/7250 Q-TOF MS, Agilent) of γ-necrodyl isobutyrate obtained from airborne collections from Nipaecoccus viridis females (FIG. 9A), and by Lavendula luisieri (Rozeira) essential oil rearrangement (FIG. 9B).

FIG. 10 presents GC-MS analyses of hydrolysis and re-esterification products of aeration collection from Nipaecoccus viridis females: natural sample of female aeration (upper panel); hydrolysis of the same sample (middle panel), and re-esterification of the same sample with isobutyric anhydride (lower panel) (analyses run on chiral Rt-βDEXsm column, temperature program: 60° C. for 1 minute then at 2° C./min to 200° C. and held for 10 minutes).

FIGS. 11A-B present schemes illustrating exemplary synthetic pathways of pheromones featuring a necrodane skeleton, according to some embodiments of the present invention.

### DESCRIPTION OF SPECIFIC EMBODIMENTS OF THE INVENTION

The present invention, in some embodiments thereof, relates to the use of a synthetic pheromone for the controlling or monitoring of mealybugs and, more particularly, mealybugs of the Nipaecoccus viridis species.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

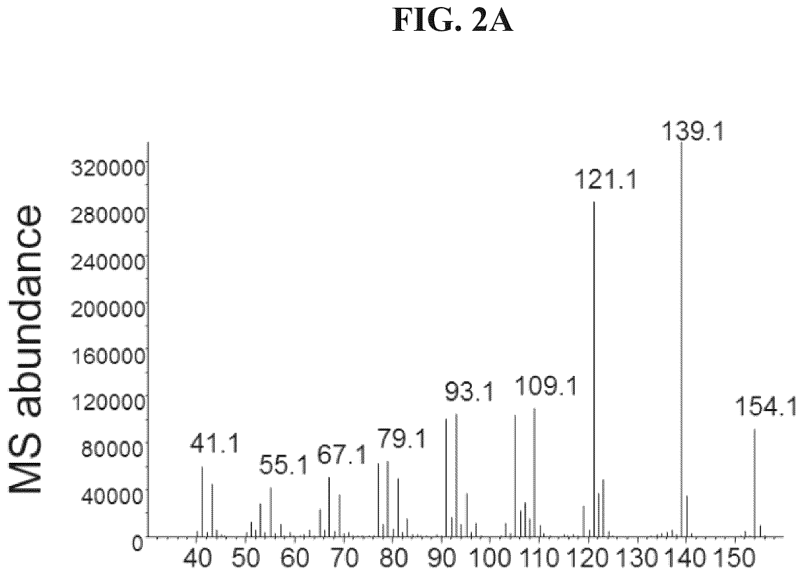

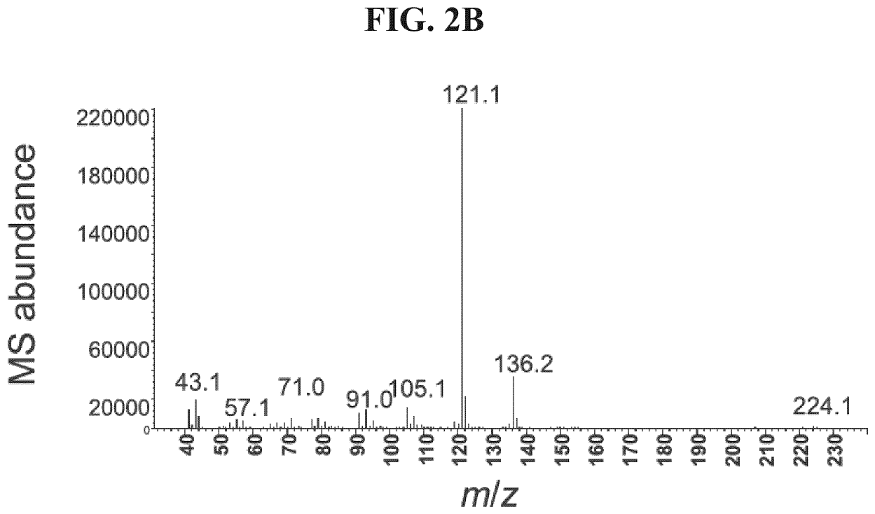

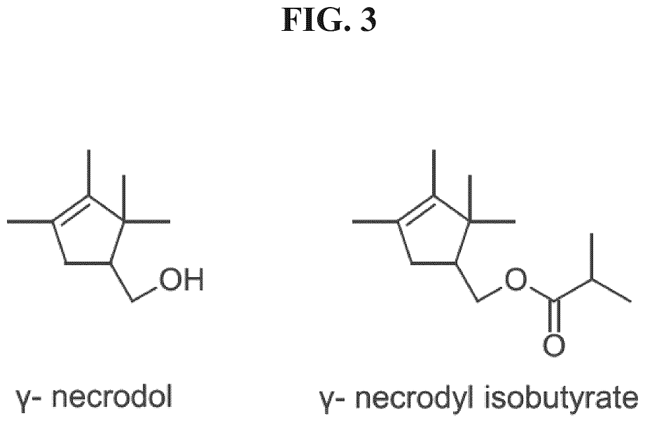

The present inventors isolated and identified the sex pheromone volatiles of N. viridis females using automated sequential SPME (solid phase micro extraction) and GC-MS (gas chromatography-mass spectrometry) analysis (SSGA). The method relies on the fact that insect pheromones are usually emitted in a circadian rhythm. Therefore, by using an auto-sampler to repeatedly collect these released volatiles by SPME fiber and then direct injection into GC-MS for characterization, the minute amounts of pheromone candidates can be revealed and distinguished from among many other volatiles that are impurities and usually not emitted in a circadian pattern. Compounds detected by SSGA were then analyzed further by GC-MS and were identified to be 2,2,3,4-tetramethyl-3-cyclopentene-1-methanol (γ-necrodol) and γ-necrodyl isobutyrate (see, FIG. 3) through synthesis and comparative analyses (see, FIGS. 2A-B, 7, 8A-B, 9A-B and 10). In order to synthesize these molecules, the present inventors converted a trans-α-necrodol and a trans-α-necrodyl acetate (which are naturally present in an essential oil of the L. luisieri species of lavender plant) to γ-necrodol, and then converted the latter to γ-necrodyl isobutyrate.

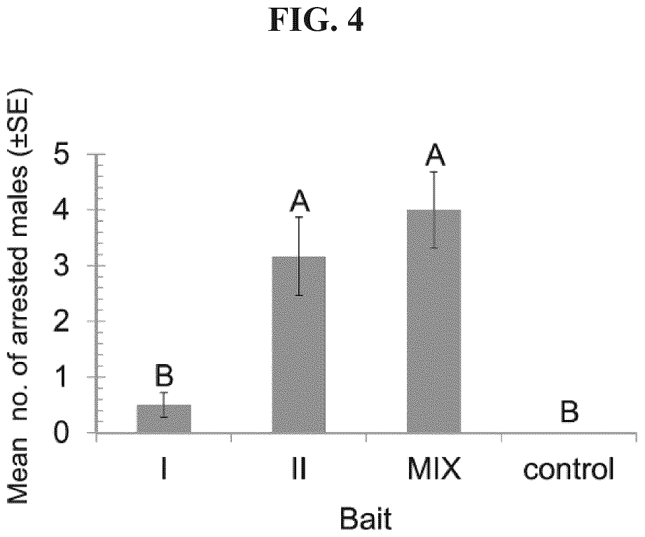

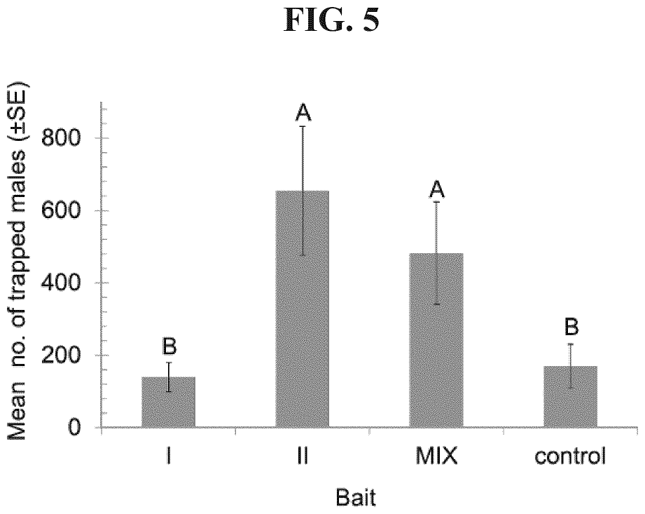

Whilst reducing the present invention to practice, the present inventors showed that males of N. viridis are attracted to the synthetic γ-necrodyl isobutyrate (see FIGS. 4 and 5). Accordingly, the present inventors propose that γ-necrodyl isobutyrate can be used for the monitoring and control of this pest and the compound simple method of synthesis provides an economically feasible method for treatment of the mealybug.

Thus, according to a first aspect of the present invention, there is provided a preparation comprising γ-necrodyl isobutyrate and an agriculturally acceptable carrier.

As used herein, the term “pheromone” refers to an attractant that is released by individual female mealybugs into the air to attract male mealybugs of the same species (e.g., downwind of the female odor source) toward the female point source.

The preparation can include, can consist essentially of, or comprise the pheromone component γ-necrodyl isobutyrate. Additional pheromone components that can be included in the preparation include γ-necrodol, and trans-α-necrodyl isobutyrate, (4,5,5-trimethyl-3-methylenecyclopent-1-en-1-yl)methyl acetate, and any other relevant mealybug pheromones.

For example, in some embodiments, the pheromone attractant composition includes a single pheromone component, such as γ-necrodyl isobutyrate. In some embodiments, the pheromone attractant composition can include a mixture of γ-necrodyl isobutyrate and trans-α-necrodol isobutyrate; or a mixture of γ-necrodyl isobutyrate and γ-necrodol; or a mixture γ-necrodyl isobutyrate and any additionally identified relevant mealybug pheromone.

The pheromone components of the preparation can interact in a synergistic manner.

In some embodiments, the preparation includes γ-necrodyl isobutyrate. The preparation can include γ-necrodyl isobutyrate in an amount of from 1%, 5% or 10% by weight, and up to 99% or even 100%, by weight, including any intermediate values and subranges therebetween, e.g., 1-99%, 1-90%, 1-80%, 1-60%, 1-50%, 1-40%, 1-30%, 1-20%, 1-10%, 10-99%, 10-90%, 10-80%, 10-70%, 10-60%, 10-50%, 10-40%, 10-30%, 10-20%, 20-99%, 20-90%, 20-80%, 20-70%, 20-60%, 20-50%, 20-40%, 50-99%, 50-90%, 50-80%, 50-70% by weight (e.g., 25% by weight, 50% by weight, or 75% by weight) to 99% by weight (e.g., 75% by weight, 50% by weight, or 25% by weight), based on the total weight of the pheromone components in the composition. In some embodiments, the preparation includes 20%-80% (e.g., 40%-80%, 60%-80%, or 70%-80%) by weight γ-necrodyl isobutyrate, based on the total weight of the pheromone components in the preparation, including any intermediate values and subranges therebetween. In some embodiments, the preparation includes only γ-necrodyl isobutyrate as a pheromone component, such that the preparation includes γ-necrodyl isobutyrate at 100% by weight, based on the total weight of the pheromone components in the preparation.

The pheromone components of the preparation, as described above, can be combined with one or more agriculturally acceptable carrier, antioxidants, and/or preservatives to form a formulation.

Examples of agriculturally acceptable carriers and formulations are disclosed in U.S. Patent Application Publication Nos. 2018/0271088, 2009/0148399, 2015/0257378, the contents of which are incorporated herein by reference.

The formulation can be in the form of a liquid (e.g., a homogeneous liquid or an emulsion), a semi-solid (e.g., a paste, a gel), or a solid (e.g., a rubber, a glass, a sol-gel).

In some embodiments, the formulation is a controlled release formulation, such that the pheromone component can be released over a period of time. Exemplary carriers for pheromone components include oils, water-in-oil emulsions or oil-in-water emulsions; a solid substrate such as fibers (e.g., cotton fibers, felts); polymers (e.g., polyethylene glycol, polymethacrylates, ethylene-vinyl acetate rubbery copolymers, poly(acrylic acid), polyolefins (e.g., polypropylene), poly(urethane), silicones, lactic and glycolic acid-based polymers, and copolymers thereof); beads (e.g., polymer beads); microcapsules (e.g., silica microcapsules); nanocapsules; glasses; a gel; and ceramics. In some embodiments, when the carrier is a solid substrate, such as fibers, polymers, microcapsules, nanocapsules; glasses, or ceramics, the pheromone attractant composition can be infused into the substrate to provide a controlled release composition. In some embodiments, a polymeric carrier can be a porous plastic substrate.

Exemplary oils to use with pheromone components include, but are not limited to, oils derived from plants such as vegetable oils and nut oils, or non-plant derived oils such as mineral oils. These are widely available and cost-effective. Formulations can include oils such as canola oil, cottonseed oil, palm oil, safflower oil, soybean oil, corn oil, olive oil, peanut oil, sunflower oil, sesame oil, nut oils, and coconut oils. Nut oils include, but are not limited to, almond oil, cashew oil, hazelnut oil, macadamia oil, mongongo nut oil, pecan oil, pine nut oil, pistachio oil, sacha inchi oil, and walnut oil. Melon and gourd seed oils are very common and inexpensive. The oils listed above include saturated, monounsaturated, and polyunsaturated fatty acids that are soluble in many compositions, especially the less polar or non-polar ones. The mineral oils are relatively inexpensive and can be used as carriers for less polar or non-polar pheromone attractant compositions. The oils can be used per se or in a form of an emulsion along with an aqueous phase.

In some embodiments, the preparation that includes the γ-necrodyl isobutyrate and optionally other pheromone components is combined with homogeneous carrier to provide composition having a desired release rate over a desired period.

As used herein, the active threshold release rate is the minimum release rate for significant insect attraction. The active threshold release rate can be determined by a dose-response test with a series of release rates ranging from very low to very high.

In some embodiments, the preparation that includes γ-necrodyl isobutyrate, in combination with one or more of γ-necrodol, trans-α-necrodyl isobutyrate and/or any other relevant pheromone component, can be separated into two or more separate formulations. For example, the pheromone attractant composition can include γ-necrodyl isobutyrate in a first formulation, and γ-necrodol in a second formulation, where the formulations can further differ in carrier and/or pheromone concentration. The two or more formulations can work together synergistically to attract male mealybugs, so long as they are placed in close proximity to (e.g., next to, or immediately next to) one another.

Exemplary preservatives that can be used in the above described preparations include, for example, sorbic acid and its salts, benzoic acid and its salts, calcium propionate, sodium nitrite, sulfites (sulfur dioxide, sodium bisulfite, potassium hydrogen sulfite, etc.) and disodium ethylenediaminetetraacetic acid (EDTA). Other exemplary preservatives include ethanol and methylchloroisothiazolinone, rosemary extract, hops, salt, sugar, vinegar, alcohol, diatomaceous earth and castor oil, citric and ascorbic acids, vitamin C, and vitamin E.

Exemplary antioxidants for use with the preparations include, but are not limited to, tocopherols (e.g., alpha-tocopherol, gamma-tocopherol, etc.), ascorbic acid, as well as synthetic antioxidants such as propyl gallate, tertiary butylhydroquinone, butylated hydroxytoluene (BHT), butylated hydroxyanisole (BHA), phenolic alcohols, flavonoids, catechins, related molecules thereof, and anthocyanins and their glycosides. The antioxidants can be soluble in most of the compositions and can react efficiently with oxygen in the dispensing systems, and therefore offer a way to decrease oxidation, breakdown, and polymerization of the pheromone attractant compositions. In some embodiments, the oxidant can also be a preservative.

While representative carriers, preservatives, and antioxidants have been listed above, it is to be appreciated that other carriers, preservatives, and antioxidants not specifically listed above can also be used.

In some embodiments, the formulation further includes a toxicant (i.e., an insecticide). Exemplary non-limiting toxicants include fipronil, boric acid, sodium tetraborate, disodium octaborate tetrahydrate, hydramethylnon, indoxacarb, dinotefuran, abamectin, fenoxycarb, spinosad, propoxur, methoprene, or any combination thereof.

The formulation can be contained in various dispensers. Non-limiting examples of dispensers include centrifuge tubes, stickpack dispensers, polyethylene bags, porous plastics, polymeric beads, rubber septa, and syringes. For example, the formulation can be absorbed into a polymeric bead or a rubber septum. The formulation can be loaded into a centrifuge tube, stickpack dispenser, or polyethylene bag.

In some embodiments, the formulation is a controlled release formulation. In some embodiments, the controlled release formulation includes γ-necrodyl isobutyrate, optionally in combination with γ-necrodol, trans-α-necrodyl isobutyrate and/or any other relevant pheromone that can slowly evaporate (i.e., volatilize) over time. In some embodiments, the controlled release formulation can alternatively or additionally be contained in a dispenser (e.g., a porous container, a porous bag) that allows the slow evaporation of the pheromone over time.

For example, the pheromone components of a given preparation can volatilize at a cumulative rate of from 0.001 mg/day (e.g., 10 mg/day, 50 mg/day, 100 mg/day, or 500 mg/day) to 1 g/day (e.g., 500 mg/day, 100 mg/day, 50 mg/day, or 10 mg/day), over a period of, for example, 3 days to 180 days (e.g., 3 days to 7 days, 3 days to 10 days, 3 days to 25 days, or 3 days to 20 days, or 30 days to 180 days, or 30 days to 120 days, or 30 days to 90 days), including any intermediate values and subranges therebetween. In some embodiment, the pheromone components of a given preparation can volatilize at a cumulative rate of from 0.1 mg/day to 1 g/day (e.g., 0.1 mg/day to 100 mg/day, 0.1 mg/day to 10 mg/day, 0.1 mg/day to 15 mg/day, 0.1 mg/day to 50 mg/day, 0.1 mg/day to 15 mg/day, 5 mg/day to 12 mg/day, 8 mg/day to 12 mg/day, 27 mg/day to 30 mg/day, 30 mg/day to 50 mg/day, 5 mg/day to 500 mg/day, 1 mg/day to 100 mg/day, 10 mg/day to 100 mg/day, or 20 mg/day to 100 mg/day) over a period of, for example, 3 days to 180 days, including any intermediate values and subranges therebetween.

According to some embodiments, the preparation or a formulation containing same is used for monitoring mealybugs. According to some of these embodiments, the pheromone components of a given preparation can volatilize at a cumulative rate of from 1 μg/day (e.g., 1 μg/day, 5 μg/day, 10 μg/day, or 50 μg/day, e.g., 1-50 μg/day, 1-40 μg/day, 10-100 μg/day, 20-100 μg/day, 30-100 μg/day, 50-100 μg/day) to 100 μg/day, including any intermediate values and subranges therebetween, over a period of, for example, 1 days to 45 days (e.g., 3 days to 7 days, 3 days to 10 days, 3 days to 20 days, or 3 days to 45 days), including any intermediate values and subranges therebetween.

A preparation or a formulation comprising same, according to the present embodiments, can be used for monitoring and/or controlling a mealybug population.

Monitoring mealybug population can be performed so as to determine, for example, the timing and/or dosing of a preparation/formulation which is aimed at mating disruption.

Monitoring can also be performed during mating disruption, so to determine timing, dosing and measurements for controlling the mealybug population.

Controlling mealybug population can be effected by means of mating disruption, which reduces the next generation population and/or reducing the population by means of, for example, mass trapping or lure and kill mealybug control methods etc., as is known in the art and in further described herein.

Herein and in the art “mating disruption” refers to a method of pest control in which a pheromone is released, optionally from several points, to a treatment area. The pheromone's release causes responding individuals (e.g., male mealybugs) to exhaust energy and die while seeking false resources or to become disoriented and unable to find mates, thus lowering reproduction and reducing subsequent populations According to some embodiments, the preparation or a formulation containing same is used for mating disruption. According to some of these embodiments, the pheromone components of a given preparation can volatilize at a cumulative rate of from 1 mg/day (e.g., 1 mg/day, 5 mg/day, 10 mg/day, or 50 mg/day, e.g., 1-40 mg/day, 10-100 mg/day, 20-100 mg/day, 30-100 mg/day, 50-100 mg/day) to 100 mg/day, including any intermediate values and subranges therebetween, over a period of, for example, 1 days to 180 days (e.g., 3 days to 10 days, 3 days to 50 days, 3 days to 100 days, 10 days to 60 days, 10 days to 100 days, 30 days to 120 days, 30 days to 120 days, 30 days to 90 days, 60 days to 120 days), including any intermediate values and subranges therebetween.

The preparations and formulations described above can be incorporated into a pest control device such as a trap. For instance, a device, e.g., dispenser, can be hang on a tree, whether in a monitoring trap or as is for mating disruption. In other instances the composition (e.g., liquid) is applied on the leaves by spraying.

Exemplary pest control devices are disclosed in U.S. patent applications having Publication Nos. 2019/0269120, 2019/0246616, 2019/0216075, 2019/0208759, the contents of which are incorporated herein.

In some embodiments, a trap is configured to capture or kill a mealybug (that is, for controlling mealybug population). The trap can include an adhesive trap (i.e., a sticky trap). In some embodiments, the mealybug trap is a non-adhesive trap, such as an electric zapper. In some embodiments, the mealybug trap can provide a source of electricity, such that mealybug can be electrocuted on contact with the electricity. In some embodiments, the trap includes one or more dispensers (e.g., a tube such as a centrifuge tube, stickpack dispenser, polyethylene bag, polymeric bead, or rubber septum) for holding and releasing pheromone attractant compositions or formulations.

In some embodiments, the trap, standalone bait, can contain one or more pheromone attractant formulations or compositions, each in a distinct dispenser. For example, the trap, bait, or bait station can include a first pheromone attractant formulation or composition that includes γ-necrodyl isobutyrate in a first dispenser. The trap, bait or bait station can further include γ-necrodol or trans-α-necrodol isobutyrate in a second dispenser. The first and second dispensers can be placed next to each another, such that the pheromone components contained in the separate dispensers can be considered to be a single composition emanating from a single point source.

According to a specific embodiment, a toxicant is included for monitoring (e.g., in funnel traps), mass trapping or lure and kill mealybug control methods. Exemplary toxicants include, but are not limited to, fipronil, boric acid, sodium tetraborate, disodium octaborate tetrahydrate, hydramethylnon, indoxacarb, dinotefuran, abamectin, fenoxycarb, spinosad, propoxur, methoprene, or any combination thereof.

As discussed above, the disclosed preparations can be used as part of a trap or standalone bait. In some embodiments, the trap containing the pheromone attractant composition is placed near areas where mealybugs (and more specifically mealybugs of the Nipaecoccus viridis species) are found, such that they may be attracted to and enclosed in the trap. The traps can be used for monitoring the dispersals and infestations of invasive mealybugs, or for killing and as mentioned controlling the mealybugs.

According to an aspect of the present invention there is provided a method of controlling a population of Nipaecoccus viridis mealybug population. According to some of these embodiments, the method comprises exposing the mealybug population to an effective amount of γ-necrodyl isobutyrate, thereby controlling the population of Nipaecoccus viridis.

As used herein “control” or “controlling” refers to decreasing the size of the mealybug population at a predetermined area (e.g., open field, greenhouse, plant or part thereof) as compared to its size in the absence of the mealybug control treatment, as described herein, over a predetermined period of time. According to a specific embodiment, the population decline is a result of mating disruption and is evident in the next generation. As mentioned, control according to some embodiments of the present invention is achieved by mating disruption due to the localized pheromone release. Alternatively, or in addition, the control is achieved by, for example, mass trapping or lure and kill methods, as described herein and in the art.

As used herein “decrease” or “decreasing” is by at least 10%, 10%, 30%, 40%, 50%, 60%, 70%, 80%, 90%, 95% or even more.

According to another aspect of the present invention there is provided a method of monitoring an amount of Nipaecoccus viridis mealybugs present in a location which is frequented by Nipaecoccus viridis:
(a) setting a trap comprising γ-necrodyl isobutyrate in said location; and
(b) determining the amount of mealybugs in said trap, thereby monitoring the amount of Nipaecoccus viridis mealybugs.

According to some embodiments, monitoring is used for controlling, as described herein, for example, by allowing to determine parameters such as the timing, dosing, area of treatment and the like, of applying the pheromone preparation/formulation, and/or of pesticides.

According to a specific embodiment, monitoring is used for determining the population density of the mealybugs, and to determine accordingly if control should be initiated. For example, if, upon monitoring, it is determined that the population density is high enough, then control is initiated by, for example, exposing the mealybugs to the pheromone preparation/formulation and/or to pesticides, and/or to cultural or biological control with natural enemies of mealybugs, or to any other mealybug control method.

According to a specific embodiment, monitoring is used to determine a timing for effecting mating disruption according to the respective embodiments (e.g., upon determining that the population density of the mealybugs is high enough), and for monitoring the mating disruption efficacy. In cases where mating disruption is successful, no mealybugs are found in monitoring traps (trap shut-down).

Determining the amount of mealybugs in the trap can be determined directly (i.e. by counting) or non-directly by weight measurement, or electronic scanning and automatic counting, and the like.

In some embodiments, a bait station including the preparation can be placed near areas where mealybugs (and more specifically mealybugs of the Nipaecoccus viridis species) frequent (e.g., is a field, vineyard or orchard where soybean, citrus, mango, tamarind and pomegranate are grown).

The pheromone component of the preparations can be synthesized from an essential oil of lavender (e.g. Lavandula luisieri, also referred to as Lavandula stoechas subsp. luisieri (Rozeira) Rozeira, or from an essential oil of any other plant that includes 10% or more by weight of a necrodol compound as defined herein. An exemplary such plant is Evolvulus alsinoides, which was reported to include more than 12% of cis-α-necrodol (Kashima and Miyazawa (2014), supra).

An essential oil is a concentrated hydrophobic liquid containing volatile (easily evaporated at normal temperatures) chemical compounds from plants. Essential oils are also known as volatile oils, ethereal oils, aetherolea, or simply as the oil of the plant from which they were extracted.

A “necrodol compound” describes a compound featuring a necrodane skeleton, as defined hereinbelow, in which R comprises a hydroxy group, and is, for example a hydroxyalkyl, preferably a hydroxymethyl. The necrodol compound can be, for example, α-necrodol, β-necrodol and/or γ-necrodol. α-Necrodol and γ-necrodol can have an R or S configuration at the position that is substituted by the hydroxy-containing group (e.g., hydroxyalkyl). The α-necrodol can also feature a cis or trans configuration.

The present inventors have found that each of γ-necrodyl isobutyrate, γ-necrodol and trans-α-necrodol isobutyrate, including enantiomers or racemates thereof, can be synthesized from the above disclosed essential oil.

According to another aspect of the present invention there is provided a method of synthesizing γ-necrodol, the method comprising subjecting an α-necrodol (trans-α-necrodol) to conditions that effect rearrangement of the α-necrodol to thereby generate γ-necrodol.

According to some of these embodiments, the conditions comprise contacting the α-necrodol with a Lewis acid.

By “Lewis acid” it is meant, as commonly accepted in the art, a compound or species which is an acceptor of a pair of electrons.

Exemplary Lewis acids that are usable in the context of the present embodiments are based on metals such as aluminum, boron, silicon, tin, titanium, zirconium, iron, copper, and zinc, which are typically substituted by one or more electron withdrawing groups, most commonly one or more halo atoms (e.g., fluoro, chloro, or bromo). Exemplary Lewis acids include BF₃, Al₂Cl₃, TiCl₄, ZnCl₂, BCl₃, and more complex Lewis acids.

In exemplary embodiments, the Lewis acid is BF₃ (e.g., in a form of BF₃ etherate).

The contacting can be performed at room temperature or at an elevated temperature.

According to any of these embodiments, the α-necrodol can be a cis-α-necrodol and/or a trans-α-necrodol.

In some embodiments, the α-necrodol is a trans-α-necrodol, and in some embodiments, the trans-α-necrodol is (−)-trans-α-necrodol.

The α-necrodol can be obtained from a potential commercial vendor, can be synthetically prepared, for example, as described in Zou et al., J. Agric. Food Chem. 2010, 58, 4977-4982, the contents of which are incorporated herein by reference, or, preferably, can be obtained from a natural source.

The present inventors have uncovered that the trans-α-necrodol can be obtained from the essential oil of lavender (e.g. Lavandula luisieri), probably as the (−) enantiomer. Similarly, cis-α-necrodol can be obtained from the same plant and from Evolvulus alsinoides (Kashima and Miyazawa 2014).

In some embodiments, the method further comprises, prior to the rearrangement reaction, processing an essential oil of lavender as described herein to thereby provide the trans-α-necrodol, as a single enantiomer or as a racemate. In some embodiments, the trans-α-necrodol is provided as (−)-trans-α-necrodol.

Preferably a distillate fraction of the essential oil of lavender (e.g. Lavandula luisieri) which comprises trans-α-necrodol and trans-α-necrodyl acetate is collected and then subjected to conditions under which the trans-α-necrodyl acetate comprised therein is converted to trans-α-necrodol. Such conditions include, for example, conditions that effect de-esterification (hydrolysis) of the trans-α-necrodol acetate, and can be, for example, conditions known to effect acid-catalyzed de-esterification or de-esterification under basic conditions (e.g., KOH and/or NaOH in a polar organic solvent such as an alcohol). The obtained trans-α-necrodol can be subjected to purification prior to the rearrangement reaction, for example, by further distillation and/or chromatography.

Optionally or in addition, a distillate fraction of the essential oil of lavender that comprises a cis-α-necrodol and/or an ester thereof is collected. If an ester is present, it is converted to the cis-α-necrodol as described above.

The obtained cis-α-necrodol can be subjected to purification prior to the rearrangement reaction, for example, by further distillation and/or chromatography.

In some embodiments, the cis-α-necrodol and the trans-α-necrodol isolated from the essential oil of Evolvulus alsinoides and lavender respectively are mixed together and subjected to the rearrangement condition, to thereby provide the γ-necrodol.

Following synthesis, the γ-necrodol can be purified—e.g. by distillation and/or chromatography (e.g., liquid chromatography).

Once purified γ-necrodol is obtained it can be converted into γ-necrodyl isobutyrate by esterification under conditions well known in the art. Isobutyric acid, an acyl halide thereof or an anhydride thereof can be used. In exemplary embodiments, γ-necrodol is reacted with isobutyric anhydride under conditions that produce γ-necrodyl isobutyrate. The γ-necrodyl isobutyrate can be optionally purified—e.g. by chromatography (e.g., liquid chromatography).

As mentioned the distillate fraction of the essential oil of lavender can also be used for synthesizing various esters of trans-α-necrodol, for example, trans-α-necrodol isobutyrate. The synthesis includes reacting the trans-α-necrodol or the mixture thereof with trans-α-necrodyl acetate, obtained upon distilling the essential oil of lavender with a selected carboxylic acid, acyl halide thereof or anhydride thereof, under conditions that effect esterification. In exemplary embodiments, trans-α-necrodol or the mixture thereof with trans-α-necrodyl acetate is reacted with isobutyryl chloride, in the presence of a base (e.g., Et₃N) and a suitable catalyst (e.g., DMAP) under conditions that produce trans-α-necrodol isobutyrate—see for example Zou et al., J. Agric. Food Chem. 2010, 58, 4977-4982, the contents of which are incorporated herein by reference.

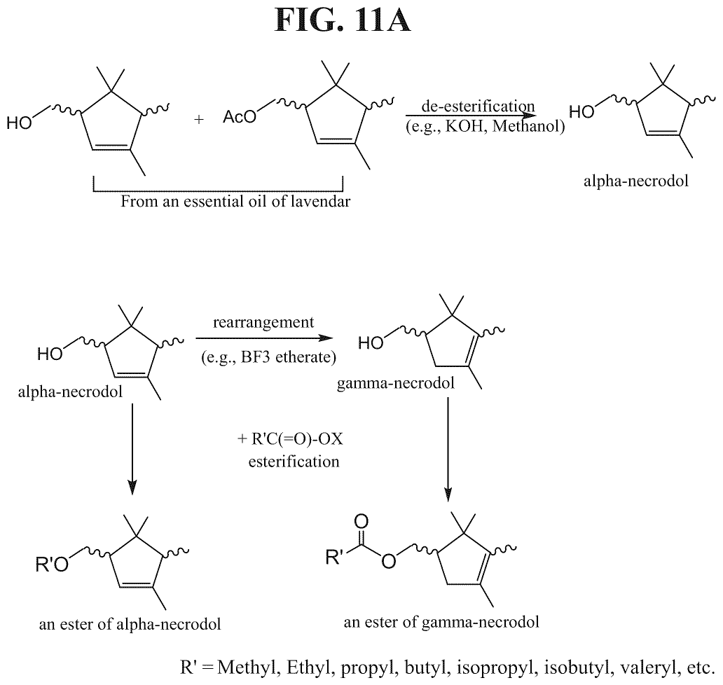

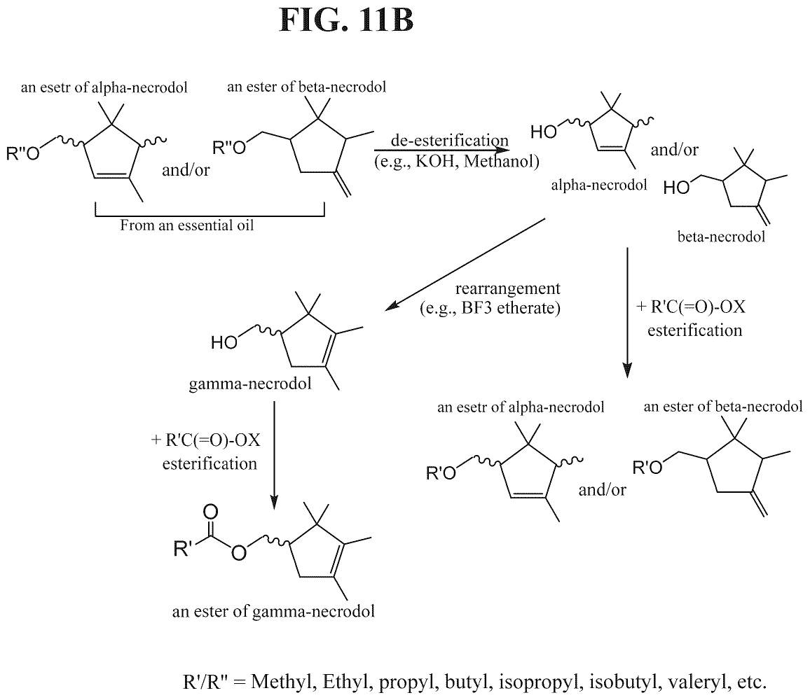

A schematic presentation of an exemplary synthesis of possible mealybug pheromones from an alpha-necrodol that can be obtained from an essential oil of, for example, lavender and optionally also from Evolvulus alsinoides, as described herein is presented in FIG. 11A. According to this presentation, esters of alpha-necrodol, gamma-necrodol and esters thereof can be prepared easily and cost-effectively.

The synthetic approach provided herein, which utilize the rearrangement and/or esterification (or trans-esterification) of a trans-α-necrodol, a cis-α-necrodol or an (e.g., acetate) ester thereof obtained from an essential oil of a lavender, can be utilized to prepare pheromones of other species of mealybugs that feature an alpha-necrodane (cis or trans), a beta-necrodane or a gamma-necrodane skeleton (e.g., as respective necrodol compounds and esters thereof).

According to an aspect of some embodiments of the present invention there is provided a method of synthesizing a mealybug pheromone featuring a necrodane skeleton, the method comprising:
subjecting a necrodol compound (e.g., trans-α-necrodol and/or cis-α-necrodol and/or (3-necrodol and/or γ-necrodol obtained from an essential oil of a plant to conditions that effect rearrangement and/or esterification of said necrodol compound, thereby synthesizing the a mealybug pheromone.

In some embodiments, the method of synthesizing a mealybug pheromone featuring a necrodane skeleton comprises:
producing a necrodol compound from an essential oil of a plant that comprises at least 10% of said necrodol compound or an ester thereof; and subjecting said necrodol compound to conditions that effect rearrangement and/or esterification of said necrodol, thereby synthesizing the mealybug pheromone.

In some of these embodiments, the plant is Lavandula luisieri and the necrodol compound is an α-necrodol, as described herein. In some embodiments, it is a trans-α-necrodol, and in some embodiments it is a single enantiomer of the trans-α-necrodol (e.g., the (−) enantiomer).

In some of any of these embodiments, producing the α-necrodol comprises converting an ester of the α-necrodol obtained from the essential oil and converting the ester to α-necrodol.

In some of these embodiments the mealybug pheromone is an ester of α-necrodol, and the mealybug pheromone is synthesized by subjecting the α-necrodol to conditions that effect esterification of the α-necrodol.

Alternatively, the necrodol compound is obtained from an essential oil of a plant that comprises a necrodol compound such as, for example, beta-necrodol or an ester thereof.

Further alternatively, an alpha-necrodol (cis and/or trans) is obtained from a plant other than lavender or other than Lavandula luisieri.

In some of these embodiments, the mealybug pheromone is an ester of such a necrodol, and is synthesized by subjecting the necrodol produced from the essential oil to conditions that effect esterification of the necrodol.

Such embodiments are presented, for example, in FIG. 11B. An essential oil of a plant that comprises 5% or more, or 10% or more, or 20% or more, by weight (based on the essential oil of the plant), of alpha-necrodol and/or beta-necrodol is used to generate these compounds (e.g., by extraction or distillation or both), and the necrodol compound(s) are subjected to esterification as described herein.

Optionally or in addition, the essential oil of the plant comprises one or more esters of the alpha-necrodol and/or beta-necrodol, and these esters are converted to the alpha-necrodol and/or beta-necrodol by de-esterification as described herein, as shown in FIG. 11B.

For example, a mealybug pheromone that is an ester of trans-α-necrodol, as described hereinabove, is obtainable, according to these embodiments, by obtaining (e.g., by means of distillation) trans-α-necrodol and trans-α-necrodyl acetate from an essential oil of Lavandula luisieri, optionally subjecting this mixture to conditions that effect de-esterification (hydrolysis) of the acetate ester to obtain trans-α-necrodol, and subjecting the obtained trans-α-necrodol to conditions that effect esterification thereof, using any carboxylate, acyl halide, or anhydride that provides the desirable ester which is the mealybug pheromone.

In some embodiments, the mealybug pheromone is an ester of gamma-necrodol.

In some of these embodiments, the essential oil of the plant comprises at least 5%, or at least 10%, or at least 20%, by weight of γ-necrodol, and the method comprises isolating the γ-necrodol and subjecting it to esterification as described herein.

Alternatively, an essential oil of a plant comprises an α-necrodol and/or a β-necrodol, or an ester thereof, as described herein, and the method comprises subjecting the α-necrodol and/or a β-necrodol (optionally upon de-esterification of an ester thereof), obtained from the essential oil, to conditions that effect rearrangement of the α-necrodol and/or a β-necrodol, to thereby obtain γ-necrodol, and optionally further subjecting the γ-necrodol to conditions that effect esterification, to thereby obtain the ester of γ-necrodol, as is also presented in FIG. 11B.

It is to be noted that whenever a necrodol, an ester thereof, or necrodane is indicated herein, it may encompass a single enantiomer thereof, if applicable, or a racemate. Typically, when produced from an essential oil of a plant, such compounds are produced as a single enantiomer, typically featuring an (−) configuration, where applicable (for example, in case of alpha-necrodols and esters thereof).

The following de-esterification (when required), rearrangement (when required) and esterification (when required) are typically such that maintain the configuration of the compound as obtained from an essential oil. In some embodiments, the method can further comprise converting an enantiomer of a necrodol, an ester thereof, or necrodane, to the other enantiomer or to a racemate, using methods known in the art.

By “necrodane structure” is it meant a monoterpene having the following skeleton:

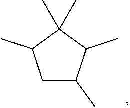

and which can be represented by the following Formula:

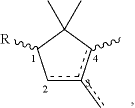

The dashed line ( [symbol] ) represents an optional double bond, which can be between the carbons at position 3 (for β-necrodanes), or between the carbons at positions 2 and 3 (for α-necrodanes), or between positions 3 and 4 (for γ-necrodanes).

The wavy lines each independently represents an R or S configuration, where applicable and/or a cis or trans configuration, where applicable.

R is typically a hydroxy-containing group, preferably a hydroxyalkyl, preferably a hydroxymethyl, or an ester thereof, but can be any other moiety, such as, but not limited to, thioalkyl, aminoalkyl, alkyl, thiol, amine, etc.

When R is a hydroxy-containing group such as hydroxyalkyl, it is referred to herein as a necrodol compound, and it provides a respective ester when reacted with R′C(═O)X (see, FIGS. 11A and 11B), with X being OH (a carboxylic acid), OZ (an ester, wherein Z is alkyl or cycloalkyl or aryl), OY with Y being a halide (e.g., chloro) (an acyl halide), or a —OC(═O)R′ (an anhydride).

R′ is the respective alkylene that provides the desirable ester and is preferably an alkyl, such as a short alkyl, of 1-8, 1-7, or 1-6 carbon atoms in length, which can be linear or branched. R′ can alternatively be an alkene, which is an alkyl as defined and described herein, which is unsaturated (e.g., having one or more double bond(s)). Examples of suitable alkylenes include, but are not limited to, methyl, ethyl, propyl, isopropyl, butyl, isobutyl, butenyl, tert-butyl, pentyl, isopentyl, isovaleryl, senecioyl, hexyl, etc.

According to some of any of the embodiments described herein, there is provided a mealybug pheromone synthesized according to the method described herein, and a preparation containing same, as described herein in any of the respective embodiments that relate to γ-necrodyl isobutyrate.

According to some of any of the embodiments described herein, there is provided a formulation that comprises the preparation of the mealybug pheromone synthesized according to the method described herein, as described herein in any of the respective embodiments, and a carrier such as an agriculturally acceptable carrier.

According to some embodiments, the mealybug pheromone preparation or formulation is used in any of the methods of monitoring and/or controlling, as described herein in any of the respective embodiments.

It is expected that during the life of a patent maturing from this application additional relevant mealybug pheromones, essential oils of plants that comprise a necrodane or necrodol compounds, and necrodol/necrodane compounds will be identified and the scope of each of these terms is intended to include all such newly uncovered components a priori.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “alkyl” describes a saturated aliphatic hydrocarbon including straight chain and branched chain groups. Preferably, the alkyl group has 1 to 30, or 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms.

The alkyl group can be an end group, wherein it is attached to a single adjacent atom, or a linking group, as this phrase is defined hereinabove, which connects two or more moieties via at least two carbons in its chain. When the alkyl is a linking group, it is also referred to herein as “alkylene” or “alkylene chain”.

Alkene and Alkyne, as used herein, are an alkyl, as defined herein, which contains one or more double bond or triple bond, respectively.

The term “cycloalkyl” describes an all-carbon monocyclic ring or fused rings (i.e., rings which share an adjacent pair of carbon atoms) group where one or more of the rings does not have a completely conjugated pi-electron system. Examples include, without limitation, cyclohexane, adamantine, norbornyl, isobornyl, and the like. The cycloalkyl group can be an end group, as this phrase is defined hereinabove, wherein it is attached to a single adjacent atom, or a linking group, as this phrase is defined hereinabove, connecting two or more moieties at two or more positions thereof.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofurane, tetrahydropyrane, morpholino, oxalidine, and the like. The heteroalicyclic group can be an end group, as this phrase is defined hereinabove, where it is attached to a single adjacent atom, or a linking group, as this phrase is defined hereinabove, connecting two or more moieties at two or more positions thereof.

The term “aryl” describes an all-carbon monocyclic or fused-ring polycyclic (i.e., rings which share adjacent pairs of carbon atoms) groups having a completely conjugated pi-electron system. The aryl group can be an end group, as this term is defined hereinabove, wherein it is attached to a single adjacent atom, or a linking group, as this term is defined hereinabove, connecting two or more moieties at two or more positions thereof.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group can be an end group, as this phrase is defined hereinabove, where it is attached to a single adjacent atom, or a linking group, as this phrase is defined hereinabove, connecting two or more moieties at two or more positions thereof. Representative examples are pyridine, pyrrole, oxazole, indole, purine and the like.

The term “halide” and “halo” describes fluorine, chlorine, bromine or iodine.

The term “haloalkyl” describes an alkyl group as defined above, further substituted by one or more halide.

The term “hydroxyl” describes a —OH group.

The term “alkoxy” describes both an —O-alkyl and an —O-cycloalkyl group, as defined herein. The term alkoxide describes —R′O— group, with R′ as defined herein.

The term “aryloxy” describes both an —O-aryl and an —O-heteroaryl group, as defined herein.

The term “thiohydroxy” or “thiol” describes a —SH group. The term “thiolate” describes a —S⁻ group.

The term “thioalkoxy” describes both a —S-alkyl group, and a —S-cycloalkyl group, as defined herein.

The term “thioaryloxy” describes both a —S-aryl and a —S-heteroaryl group, as defined herein.

The “hydroxyalkyl” is also referred to herein as “alcohol”, and describes an alkyl, as defined herein, substituted by a hydroxy group.

The term “acyl halide” describes a —(C═O)R″″ group wherein R″″ is halide, as defined hereinabove.

The term “carboxylate” as used herein encompasses C-carboxylate and O-carboxylate.

The term “C-carboxylate” describes a —C(═O)—OR′ end group or a —C(═O)—O— linking group, as these phrases are defined hereinabove, where R′ is as defined herein.

The term “O-carboxylate” describes a —OC(═O)R′ end group or a —OC(═O)— linking group, as these phrases are defined hereinabove, where R′ is as defined herein.

A carboxylate can be linear or cyclic. When cyclic, R′ and the carbon atom are linked together to form a ring, in C-carboxylate, and this group is also referred to as lactone. Alternatively, R′ and O are linked together to form a ring in O-carboxylate. Cyclic carboxylates can function as a linking group, for example, when an atom in the formed ring is linked to another group.

The term “thiocarboxylate” as used herein encompasses C-thiocarboxylate and O-thiocarboxylate.

The term “C-thiocarboxylate” describes a —C(═S)—OR′ end group or a —C(═S)—O— linking group, as these phrases are defined hereinabove, where R′ is as defined herein.

The term “O-thiocarboxylate” describes a —OC(═S)R′ end group or a —OC(═S)— linking group, as these phrases are defined hereinabove, where R′ is as defined herein.

A thiocarboxylate can be linear or cyclic. When cyclic, R′ and the carbon atom are linked together to form a ring, in C-thiocarboxylate, and this group is also referred to as thiolactone.

Alternatively, R′ and O are linked together to form a ring in O-thiocarboxylate. Cyclic thiocarboxylates can function as a linking group, for example, when an atom in the formed ring is linked to another group.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Insects. N. viridis populations were collected from two citrus groves in Israel, in West Negev and the Galilee areas. These populations were used to establish a colony on sprouted potatoes (Solanum tuberosum) in a rearing room at 25±1° C., 50-60% relative humidity, and under a photoperiod of 14L:10D. First stage larval instars were separated to a different cage and emerged adult males were removed manually each day (Levi-Zada et al. 2014). Adult females, 10 days after the last molt, were transferred to clean potato sprouts for further use in experiments.

Isolation of Sex Pheromone and Circadian Rhythm of Release. In order to reveal compounds released in a circadian rhythm that would be considered as candidates of sex pheromone components, an automated SSGA was conducted with 300-500 virgin females on potato sprouts placed in a 250 ml amber vial on a commercial GC-MS auto-sampler tray (MPS2-Twister, Gerstel, Germany). The auto-sampler was equipped with a SPME syringe and a 65 μm polydimethylsiloxane/divinylbenzene fiber (Supelco, Sigma-Aldrich, Israel). The auto-sampler was programmed by software (Maestro, Gerstel, Germany) to collect volatiles emitted by the females every 2 hours over a few days and inject each sample immediately into the GC-MS injection port. The fiber was cleaned prior to each sequence by baking in the needle heater unit of the auto-sampler for at least 10 min at 240° C. and then between volatile samplings by the GC-MS injector port (250° C. for 6 min). The auto-sampler was located close to a window. Lights in the room were turned off at 20:00 PM and turned on the next day at 6:00 AM to simulate a natural daylight cycle.

Airborne Volatile Collections from Females. Airborne collections were done by passing charcoal-filtered air through a 500 ml wide neck bottle with Drechsel bottle head that contained a few potatoes with sprouts infested with 300 to 1500 virgin females. The airborne volatiles were trapped in a 4 mm ID×10 cm long glass tube filled with SuperQ (Altech, IL, USA). Aerations were done during each of three days, between 15:00-20:00 PM, when pheromone candidates were released according to SSGA, in order to reduce background contamination by non-relevant peaks originating from the potatoes. After each volatile collection, the SuperQ in the glass tube was washed with 1 ml of n-hexane and 100 μl of this solution was concentrated to 20 μl for further analyses.

GC-MS Analysis of Samples. Analyses of volatiles was performed on a non-polar Rxi®5SilMS (Restek, PA, USA) column (30 m×0.25 mm ID×0.25 μm film) in an Agilent 6890N/5973 GC-MS instrument equipped with a commercial auto-sampler (MPS2-Twister, Gerstel, Germany) that was programmed by Maestro software 1.4.8.14 (Gerstel, Germany). The column was kept at 50° C. for 5 min, then programmed at 10°/min to 230° C. and held for 10 min. Analyses on polar VF-23 (Varian Inc., Lake Forest, CA) column (30 m×0.25 mm ID×0.25 μm film) and chiral column Rt-βDEXsm (Restek, PA, USA) were performed using an Agilent 7890A GC interfaced with an Agilent 5975C MS detector and flame ionization detector (FID) with a commercial auto-sampler (GC-sampler 80, Agilent Technologies, Switzerland), that was operated by MassHunter GC-MS Acquisition B.07.02.1938 software (Agilent, USA). The polar column was kept at 50° C. for 5 min, then programmed at 10°/min to 230° C. and held for 10 min. The chiral column was kept at 60° C. for 1 min, then programmed at 2°/min to 200° C. and held for 20 min. Column helium flow was 1.5 ml/min and it was split to both detectors equally by an Agilent purged two-way effluent splitter, enabling qualitative and quantitative analyses simultaneously. Analyses on both machines were performed in the splitless mode with the split valve opened after 1 min, the MS m/z range was 40-350 a.m.u. and the inlet temperature was kept at 230° C. A 4 mm ID liner was used for liquid injections and was replaced with a 0.75 mm ID glass inlet liner for SPME injections. Ten μl syringe was used for liquid analyses. Wiley 8 and personal GC-MS libraries were used for structure elucidation. High resolution (HR)-GC-MS analysis (7890B/7250 Q-TOF GC-MS, Agilent) in an electron impact (70 eV) mode was performed on HP 5MS ultra column (15 m×250 mm×0.25 μm, Agilent) that was kept at 60° C. for 3 min, then programmed at 15°/min to 230° C. and held for 15 min. Column helium flow was 1 ml/min.

Chemicals. Lavender (Lavandula luisieri) essential oil was purchased from Eden Botanicals, CA, USA. Standards of 2-isopropyliden-5-methyl-4-hexen-1-yl butyrate (isolavandulyl butyrate), trans-3,4,5,5-tetramethyl-2-cyclopentene-1-methanol (trans-α-necrodol), cis-3,4,5,5-tetramethyl-2-cyclopentene-1-methanol (cis-α-necrodol), trans-2,2,3-trimethyl-4-methylene-cyclopentamethanol, (trans-β-necrodol) and cis-2,2,3-trimethyl-4-methylene-cyclopentamethanol (cis-β-necrodol) were used to formulate Table 1 below. BF₃·Et₂O (Sigma-Aldrich, Israel) was dried overnight on CaH2 powder (Acros, NJ, USA), then, prior to use, dry ether (20% v/v, dried on Na/benzophenone) was added and the reagent was distilled from the mixture. All other reagents were purchased from Sigma-Aldrich (Rehovot, Israel) and used without purification, unless otherwise indicated.

Behavioral Bioassays. The two putative female sex pheromone components and their 1:1 mixture were tested in 14-cm diameter glass Petri dish arenas (Mendel et al. 2012). Four filter paper disks (5-mm diameter, Whatman No. 1) impregnated with 1 μl n-hexane solutions containing 10 ng γ-necrodol (I), 10 ng γ-necrodyl isobutyrate (II), 5 ng of each I+II combined, and solvent only (control) were equally spaced in random order on the periphery of the arena in each test (n=6). Twenty-four males were released in each of the tests at the center of a Petri dish and after 1 h the number of males found on each paper disk was recorded. A non-parametric Kruskal-Wallis rank sum test (PMCMR package R-Statistics 2.3.2) was used to determine whether treatments differed significantly in arrestment of males. If a significant difference was found then post-hoc pairwise Conover tests with Bonferroni correction for multiple comparisons was performed (same PMCMR package).

Flight bioassays of males compared the attractiveness of the female produced components in a 3×3 m² rearing room in which a colony of mealybugs, in several open plastic containers, was placed on the floor in the center. Traps consisting of white cards (18×10 cm²) covered with 80% polyisobutene adhesive (Rimifoot, Rimi, Petah Tikva, Israel) were baited with 0.5 ml 5×8 mm ID×OD polyethylene vials (Just Plastic, Norfolk, UK) containing either 20 μg of γ-necrodol (I), γ-necrodyl isobutyrate (II), their mixture (I+II, 20 μg each), each in 200 μl n-hexane solution, or only 200 μl n-hexane control. The baited traps were hung at 1.2 m height, separated by 2.8 m and were rotated in position every day for 12 days. The Kruskal-Wallis rank sum test and post-hoc Conover tests above were used to compare catches on treatments for statistical differences.

### Example 1

### Isolation and Identification of Pheromones

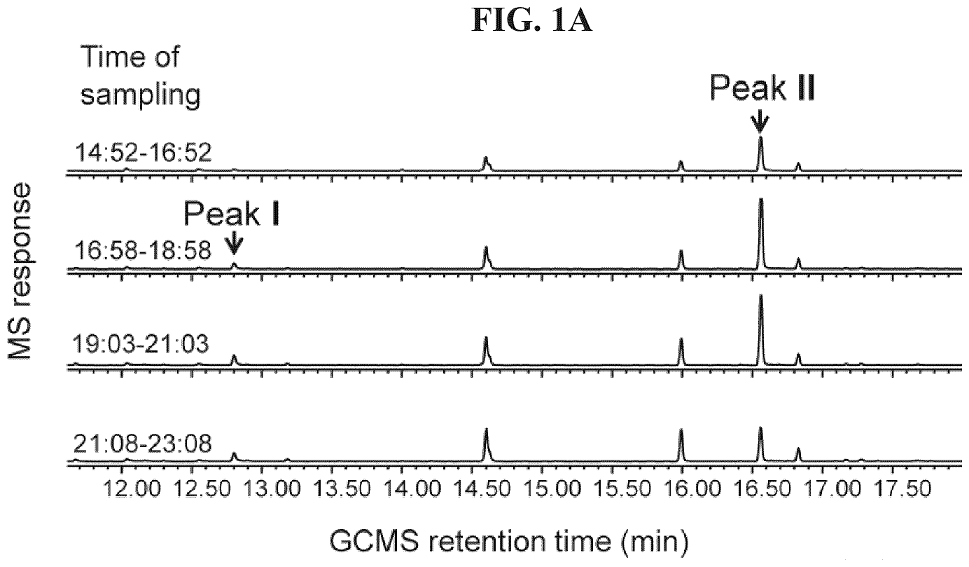

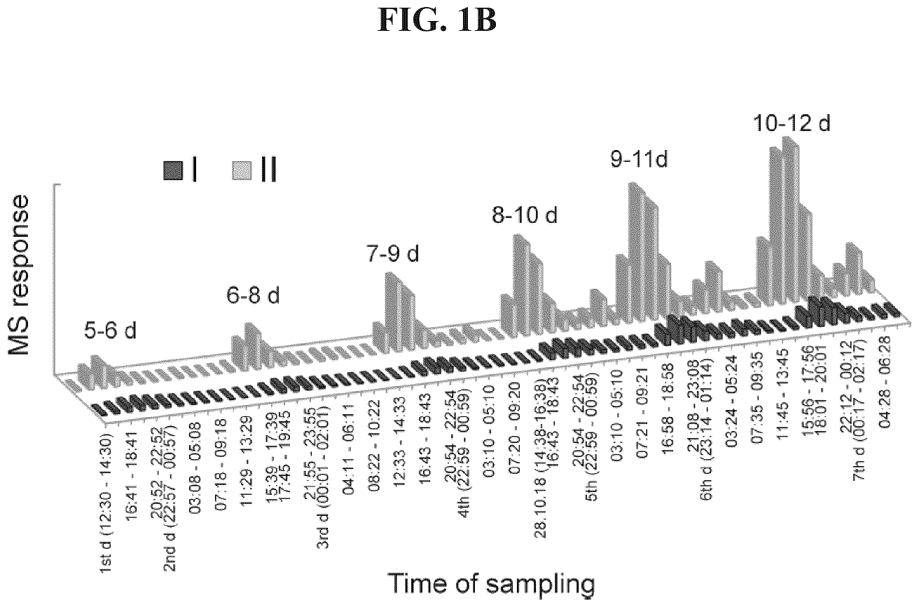

The results obtained from the sequential sampling analysis (FIGS. 1A and 1B) clearly show that the spherical mealybug females released two compounds at only certain times on a circadian cycle, peaking at 17:00 PM±2 h, about eleven hours into the photophase. The amounts released of the two compounds increases ˜6.8 fold with the age of the female from day 5 to day 10 after last molt (FIG. 1B).

The retention index (RI), on three different columns, of the two compounds were compared to those of mealybug pheromones known in the literature and to compounds that have structural features similar to these known phermones, as shown in Table 1.

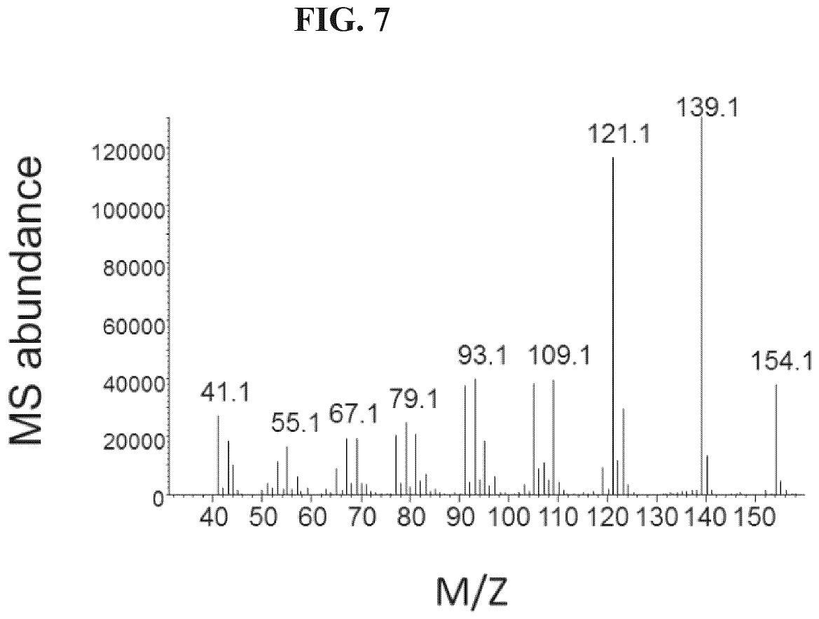

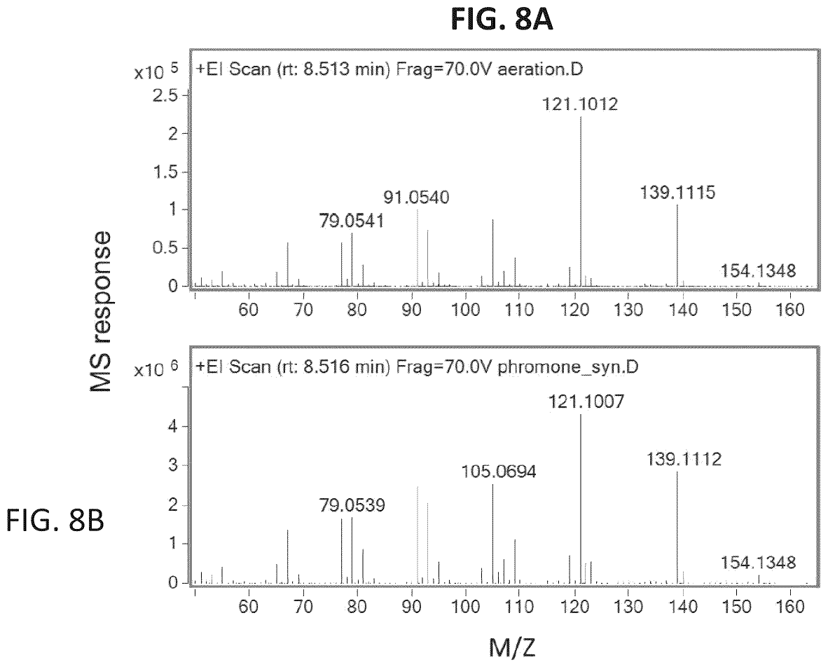

Compound I emitted in a circadian rhythm (FIG. 1A) has a retention index (RI) of 1184 (Table 1) on a non-polar Rxi®5SilMS column (Restek, PA, USA) and characteristic fragment ions of m/z (EI, 70 eV): 55(14), 67(15), 77 (18), 79 (18), 81 (15), 91 (28), 93 (32), 105 (29), 109 (36), 121 (89), 123 (14), 139 (100), 154 (29), as shown in FIGS. 2A and 7. Compound II has an RI of 1448 on the same column (Table 1) and its characteristics ions are: 55 (2), 71 (2), 77 (3), 79 (4), 81 (2), 91 (6), 93 (6), 105 (7), 107 (4), 121 (100), 122 (12), 136 (18), 224 (1), as shown in FIGS. 2B and 8A. The major compound, peak II, that consistently increased in the afternoons (see, FIG. 1B) has a mass spectrum that is very similar to the MS of two other mealybug pheromones that are known in the literature. They comprise irregular monoterpenoids: isolavandulyl butyrate of the Japanese mealybug Planococcus kraunhiae (Sugie et al. 2008) and trans-α-necrodyl isobutyrate of the grape mealybug Pseudococcus maritimus (Figadere et al. 2007). However, under the same GC-MS conditions a synthetic standard of isolavandulyl butyrate displays an RI of 1496 (Table 1), and thus cannot represent peak II in FIGS. 1A-B.

Table 1 below presents the retention indexes of different necrodols and conjugated esters on three different GC columns (30 m×0.25 mm id×0.25 μm film): non-polar Rxi®5silms column (restek, PA, USA), polar VF-23 ms (Varian, CA, USA) and chiral Rt-βDEXsm (restek, PA, USA).

TABLE 1

| | Retention Index (RI) | | |
|---|---|---|---|
| Compound | Rxi ®5SiIMS | VF-23ms | Rt-βDEXsm |
| I (γ-Necrodol)ª | 1184 | 1731 | 1344 |
| II (γ-Necrodyl isobutyrate)ª | 1448 | 1774 | 1471 |
| γ-Necrodol | 1184 | 1731 | 1344 |
| γ-Necrodyl isobutyrate | 1448 | 1774 | 1471 |
| γ-Necrodyl butyrate | 1491 | 1854 | 1521 |
| Isolavandulyl butyrate | 1496 | 1864 | 1524 |
| Trans-α-necrodol | 1152 | 1673 | 1319 |
| Cis-α-necrodol | 1161 | 1695 | 1289 |
| Trans-β-necrodol | 1202 | 1829 | 1360 |
| Cis-β-necrodol | 1214 | 1857 | 1368 |
| Trans-α-necrodyl isobutyrate | 1421 | 1747 | 1441 |
| Cis-α-necrodyl isobutyrate | 1434 | 1763 | 1452 |
| Trans-β-necrodyl isobutyrate | 1451 | 1823 | 1475 |
| Cis-β-necrodyl isobutyrate | 1475 | 1826 | 1500 |
| γ-Necrodyl acetate | 1285 | 1655 | 1314 |

^{a}Natural, from N. viridis females volatile collections. All other compounds are synthetic.

Compound I with an RI of 1184 and a molecular ion mass fragment of 154 m/z was assumed, based on the GC-MS library, to be the terpene 3,4,5,5-tetramethyl-2-cyclopentene-1-methanol (α-necrodol). Comparison of the RI and MS of compound I with those of trans-α, cis-α and trans-β and cis-β necrodols showed that they display different RI's of 1152, 1161, 1202 and 1214, respectively (Table 1), and thus indicated that compound I is a different necrodol isomer. Comparing the MS of compound I with the literature (Jacobs et al. 1990; Pamingle et al. 1991) suggested that it is γ-necrodol (2,2,3,4-tetramethyl-3-cyclopentene-1-methanol) (FIG. 3). In addition, though the RI of γ-necrodol is unknown in the literature, it was reported that γ-necrodol elutes on DB-5 non-polar column between trans-α-necrodol and trans-β-necrodol (Zou et al. 2010). Thus, since compound I has RI of 1184, which is eluting between trans-α-necrodol (RI=1152) and trans-β-necrodol (RI=1202) on non-polar Rxi®5SilMS column, compound I seems to be γ-necrodol.

γ-necrodol was produced by rearrangement of trans-α-necrodol distilled from the essential oil of Lavandula luisieri, as described in Example 3 below.

The product of the rearrangement reaction, γ-necrodol, co-elutes with compound I on non-polar Rxi®5SilMS column and both have identical MS, as shown in FIGS. 2A, 7 and 8A-B. γ-Necrodol also co-elutes with compound I that was collected in female aeration samples on polar VF-23 ms (Varian, CA, USA) and chiral Rt-βDEXsm (Restek, PA, USA) columns, with RI's of 1731 and 1344, respectively (Table 1). These data therefore confirmed that compound I is γ-Necrodol.

It is known that mealybug pheromones are usually carboxylic esters of monoterpene alcohols with irregular non-head-to-tail linkages (Sugie et al. 2008). Compound II, according to molecular ion mass fragments (M+=224 m/z) and a mass fragment (M+−C₄H₉OOH=136 m/z) shown in FIG. 2B, is an ester of a 4 carbon carboxylic acid, either butyric or isobutyric acid. This compound seems to be the putative pheromone component emitted by N. viridis females in a circadian rhythm (FIGS. 1A-B) with its alcohol precursor γ-necrodol (FIG. 3).

After γ-necrodol was identified as compound I, it was reacted with isobutyric and butyric anhydride (in pyridine). The two resulting esters were analyzed by GC-MS on the non-polar, polar and chiral columns above. The analyses showed that γ-necrodyl isobutyrate and compound II co-elute and have identical MS. RI's of γ-necrodyl isobutyrate on these columns are 1448, 1774, and 1471, respectively (Table 1). The RI's of γ-necrodylbutyrate on these columns are 1491, 1854, and 1521, respectively (Table 1) and therefore this isomeric ester is not compound II.

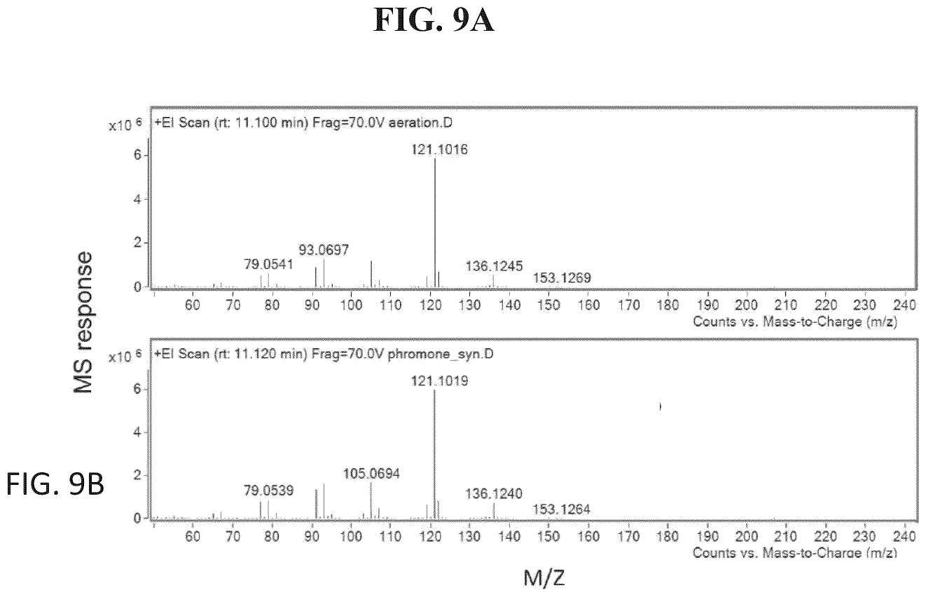

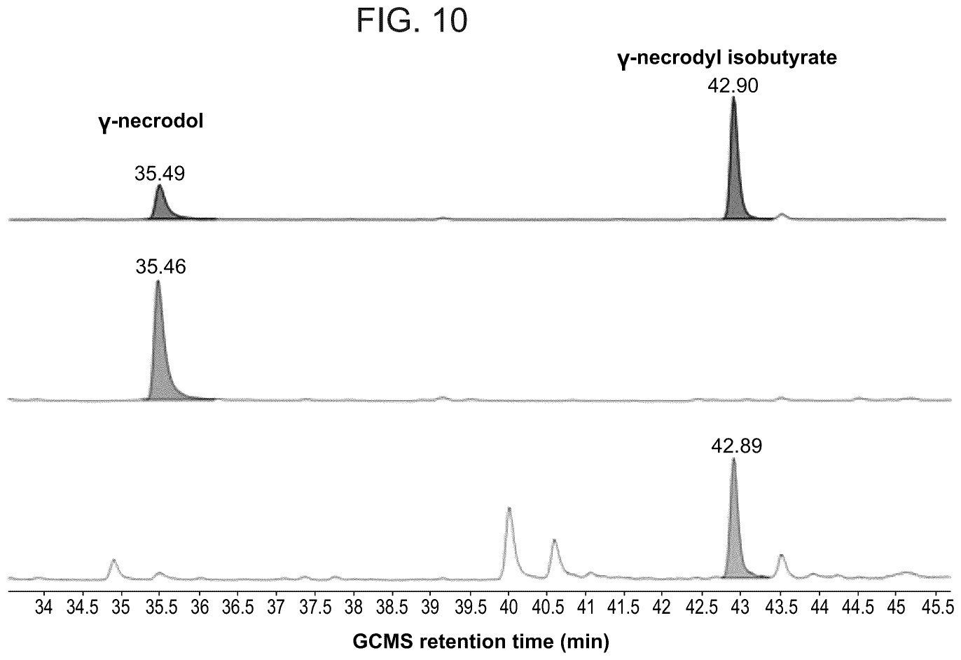

HR-GC-MS analysis of compound I in a sample of female aeration gave a molecular peak of 154.1348 m/z (FIG. 8A) which refers to a molecular formula of C₁₀H₁₈O and was identical to that of the synthetic γ-necrodol (FIG. 8B). HR-GC-MS analysis of compound II in the female aeration sample did not display the molecular ion (FIG. 9A). However, the Rt (retention time) and HR mass spectrum of T-necrodyl isobutyrate in the natural sample were identical with the synthetic sample (FIG. 9B). In addition, hydrolyzation of an aliquot of the female aeration sample by LiAlH₄ (in ether) resulted in the disappearance of peak U (v-necrodyl isobutyrate) and increased the size of peak I (γ-necrodol) in GC-MS analysis (FIG. 10, upper and middle panels). Re-esterification of peak I by isobutyric anhydride (in ether/pyridine) gave peak II exclusively (FIG. 10, lower panel).

Analyses of eight samples from female aerations on a polar column connected to GC with MS and FID detectors running in parallel showed that the actual ratio between γ-necrodol and γ-necrodyl isobutyrate is 44:56 (±1.76% SE).

As described hereinabove, the isolation of a mealybug pheromone using the “classical” methods of solvent extraction or adsorbent extraction is very complicated.

Here it is shown that by using the automatic sequential SPME/GC-MS sampling analysis (SSGA) technique previously described for a moth pest (Levi-Zada et al. 2011), is advantageous over traditional isolation methods and enabled isolating and identifying the natural pheromone components of the spherical mealybug pest from only a few hundred virgin females, much less than in previous mealybug studies.

### Example 2

### Bioassays

In the Petri dish bioassay baits with compound II attracted a mean of 3.16±0.7 (±SE) males, baits with compound I attracted 0.5±0.2, baits with a mix of compounds I and II had 4±0.7, and none were found on the empty paper disk. A Kruskal-Wallis rank sum test gave chi-square=18.62, df=3, P=0.0004, indicating the treatments differed significantly in arrestment of males. Post-hoc pairwise Conover tests with Bonferroni correction for multiple comparisons indicated that II or I+II were significantly different from I or control (all P<0.0001), however II was not different from I+II, nor were I and control different (FIG. 4).

In the flight bioassay, bait II caught an average of 654±178 (±SE) males per day and bait I+II caught 482±142 males per day, but both these baits attracted significantly more than either the bait treated with compound I (139±41) or the control (169±61). Catches per day of bait II and mix of I+II were not significantly different, while catches of bait I and control were not significantly different (Kruskal-Wallis P=0.001 and Conover's test above at α=0.05) (FIG. 5).

The results of the four-choice Petri dish bioassays suggest that γ-necrodyl isobutyrate is more attractive to N. viridis males than γ-necrodol. The flight tests of males also suggest that γ-necrodyl isobutyrate, is the more attractive pheromone component. The catches on controls in the room were considerable, possibly due to the relatively still air and effects of natural pheromone release from females in the rearing boxes.

The pheromone components described herein (e.g., γ-necrodyl isobutyrate, γ-necrodol) are further tested in field bioassays as follows:

Small delta sticky traps are applied in all tests with the exception of the test comparing different trap types. The traps are baited with a dispenser (rubber or polyethylene) loaded with 1 mg of each pheromone component and the mixture dissolved in n-hexane. BHT (Sigma, 5%) is added to all blends in test evaluating the activity of the pheromone components. Baited traps and unbaited (empty) traps are hung on the trees at a height of 1.5 m above the ground. Treatments are replicated five times in a randomized block design with 20 m between traps and rotated one position after each count (approximately every week) during the experiment.

The traps are sampled periodically to determine the amount and/or level of captured males that refer to population density, the phenology of male's flight and the pest areal distribution.

### Example 3

### Chemical Syntheses

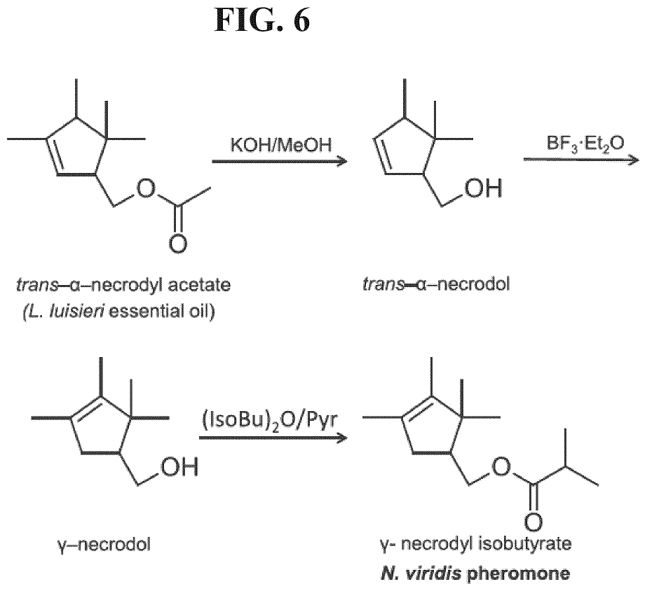

Garcia-Vallejo et al. (1994) found that the main constituents in the essential oil of Lavandula luisieri are trans-α-necrodol and trans-α-necrodyl acetate. Pamingle et al. (1991) showed that α-necrodol or β-necrodol can be converted by Lewis acid rearrangement to γ-necrodol with the double bond moving to a thermodynamically preferred position (see, FIG. 6).

The present inventors have therefore devised and successfully practices a new synthetic pathway for preparing γ-Necrodol from the essential oil of Lavandula luisieri.

γ-Necrodol was synthesized by rearrangement of trans-α-necrodol and trans-α-necrodyl acetate that were distilled from L. luisieri essential oil, as previously described.

The distillate was hydrolyzed with KOH (2M) in methanol. After usual workup, the alcohol (trans-α-necrodol, 49% chemical purity) was isomerized by BF₃-Et₂O (following the procedure described in Pamingle et al. 1991). The reaction was followed by gas chromatography until no residue of trans-α-necrodol was observed and the final product was purified by distillation (37° C./0.8 mm Hg). The yield of the final product γ-necrodol was 15% (from L. luisieri essential oil) and chemical purity of 88%. Higher chemical purity (98-99%) was achieved by liquid chromatography on AgNO₃ (10%)/SiO₂ glass column covered with aluminum foil, using 3% ether in pentane as elution solvent.

M/Z (EI, 70 eV): 55(14), 67(15), 77 (18), 79 (18), 81 (15), 91 (28), 93 (32), 105 (29), 109 (36), 121 (89), 123 (14), 139 (100), 154 (29).

¹H NMR (CDCl₃; 600 MHz) δ (in ppm): 3.78 (dd, J=5.7, 10.6, 2H), 3.62 (dd, J=7.6, 10.6, 1H), 2.30 (m, 1H), 1.99 (m, 2H), 1.59 (s, 3H), 1.47 (s, 1H), 1.04 (s, 3H), 0.82 (s, 3H. ¹³C NMR (CDCl₃; 600 MHz) δ (in ppm): 139.05, 128.44, 64.83, 50.85, 47.8, 39.66, 27.58, 20.37, 14.55, 9.52.

γ-Necrodyl isobutyrate was synthesized from γ-necrodol obtained above (1 gram, 6.4 mmol, 88% chemical purity) by reacting with isobutyric anhydride (1.4 ml, 8.4 mmol) in pyridine (1 ml). The reaction mixture was stirred overnight and then poured into cold 1M HCl (10 ml). The product was extracted three times with 15 ml of n-hexane, washed with 20 ml of aqueous NaHCO₃ and dried over MgSO₄. The solvent was evaporated and the residue was chromatographed on a column of AgNO₃ (10%)/SiO₂ covered with aluminum foil, using n-hexane as eluent to give 0.78 gram of γ-necrodyl isobutyrate in a yield of 11% based on L. luisieri essential oil. Elementary analysis of the synthetic γ-necrodyl isobutyrate (synthesized from 98% γ-necrodol mentioned above) confirmed the molecular formula of C₁₄H₂₄O₂: C 74.76% (calc. 74.94%) H 10.49% (calc. 10.78%).

M/Z (EI 70 eV, Agilent 5975C): 55 (2), 71 (2), 77 (3), 79 (4), 81 (2), 91 (6), 93 (6), 105 (7), 107 (4), 121 (100), 122 (12), 136 (18), 224 (1) (see, FIG. 8B).

¹H NMR (CDCl₃; 600 MHz) δ (in ppm): 4.08-4.15 (m, 2H), 2.54 (sep, J=7, 1H), 2.23 (m, 1H), 2.11 (qui, J=7.8, 1H), 1.98 (m, 1H), 1.58 (s, 3H), 1.47 (s, 3H), 1.17 (d, J=0.7, 3H), 1.16 (d, J=0.7, 3H), 1.05 (s, 3H), 0.81 (s, 3H).

¹³C NMR (CDCl₃; 600 MHz) δ (in ppm): 177.69, 138.91, 128.23, 65.98, 48.02, 47.06, 39.40, 34.47, 27.36, 20.29, 19.38, 19.35, 14.46, 9.58.

The GC-MS analyses on chiral column gave only one peak of trans-α-necrodol for L. luisieri essential oil, and only one peak for γ-necrodol in aerations of N. viridis mealybug. Therefore, it is assumed that each is only one respective enantiomer. Thus, the synthesis described herein produced only one enantiomer of γ-necrodol and γ-necrodyl isobutyrate, which are the same as in the N. viridis mealybug, and each was determined to be the (−) enantiomer. The stereoconfiguration was determined by dissolving the pure compound in chloroform at a concentration of about 1 gram/100 cm³ and testing specific optical rotation using a polarimeter.

It is noted that a racemic mixture or (+) enantiomers can be produced from respective components, in cases that these components are present in a plant, or can be generated by methods known in the art for racemization and/or enantioselective syntheses, from respective (−) enantiomers that produced from plants.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

### BIBLIOGRAPHY

Bergmann J, Tapia J, Bravo M, Zaviezo T, Flores M F (2019) Synthesis of citrophilus mealybug sex pheromone using chrysanthemol extracted from Pyrethrum (Tanacetum cinerariifolium). Nat Prod Res 33:303-308. doi:10.1080/14786419.2018.1446136
Eisner T, Meinwald J (1982) Defensive spray mechanism of a silphid beetle (Necrodes surinamensis). Psyche. 89: 357-367. doi:10.1155/1982/41643
El-Sayed A M (2019) The Pherobase: Database of Pheromones and Semiochemicals. www(dot)pherobase(dot)com
Figadere B A, McElfresh J S, Borchardt D, Daane K M, Bentley W, Millar J G (2007) trans-α-Necrodyl isobutyrate, the sex pheromone of the grape mealybug, Pseudococcus maritimus.
Tetrahedron Lett 48: 8434-8437. doi:10.1016/j.tetlet.2007.09.155 Gerson U, Applebaum S (2015) Plant pests of the Middle East. www(dot)agri(dot)huji(dot)ac(dot)il/mepests/entry/Pseudococcidae
Griffiths K, Derksen A (2010) 2009-2010 CAPS Surveys for Nipaecoccus viridis (Newstead) (Hemiptera: Pseudococcidae) in South Florida. Division of Plant Industry, Florida Dept. Agric. Consumer Serv. Gainsvile, Florida.
Garcia-Vallejo M I, Garcia-Vallejo M C, Sanz J, Bernabe M, Velasco-Negueruela A (1994) Necrodanes derivatives in Lavandula luisieri, new compounds to the plant kingdom. Phytochemistry 36: 43-45. doi:10.1016/S0031-9422(00)97009-2
Jacobs R T, Feutrill G I, Meinwald J (1990) Defense mechanisms of arthropods. 84. Synthesis of (−)-α-necrodol and (−)-β-necrodol: Novel cyclopentanoid terpenes from a carrion beetle. J Org Chem 55: 4051-4062. doi: 10.1021/jo00300a021
Kashima and Miyazawa, Chemistry & Biodiversity, Vol. 11, page 396 (2014)
Levi-Zada A, Fefer D, Anshelevitch L, Litovsky A, Bengtsson M, Gindin G, Soroker V (2011) Identification of the sex pheromone of the lesser date moth, Batrachedra amydraula, using sequential SPME auto-sampling. Tetrahedron Lett 52: 4550-4553. doi:10.1016/j.tetlet.2011.06.091
Levi-Zada A, Fefer D, David M, Eliyahu M, Franco J C, Protasov A, Dunkelblum E, Mendel Z (2014) Diel periodicity of pheromone release by females of Planococcus citri and Planococcus ficus and the temporal flight activity of their conspecific males. Naturwissenschaften 101: 671-678. doi: 10.1007/s00114-014-1206-y
Mendel Z, Jasrotia P, Protasov A, Kol-Maimon H, Levi-Zada A, Franco J C (2012) Responses of second-instar male nymphs of four mealybug species (Hemiptera: Pseudococcidae) to conspecific and heterospecific female sex pheromones. J Insect Behav 25: 504-513. doi:10.1007/s10905-012-9317-9
Pamingle H, Snowden R L, Schulte-Elte K H (1991) 52. Stereoselective conversion of campholene to necrodane-type monoterpenes. Novel Access to (−)-(R,R)— and (R,S)-α-necrodol and the enantiomeric γ-necrodols. Helv Chim Acta 74: 543-548. doi:10.1002/hlca.19910740310
Sharaf N W, Meyerdirk D E (1987) A review on the biology, ecology and control of Nipaecoccus viridis (Homoptera: Pseudococcidae). Miscellaneous Publications of the Entomological Society of America 66: 1-18.
Sugie H, Teshiba M, Narai Y, Tsutsumi T, Sawamura N, Tabata J, Hiradate S (2008) Identification of a sex pheromone component of the Japanese mealybug, Planococcus kraunhiae (Kuwana). Appl Entomol Zool 43: 369-375. doi:10.1303/aez.2008.369
Tabata J, Teshiba M, Shimizu N, Sugie H (2015) Mealybug mating disruption by a sex pheromone derived from lavender essential oil. J Essent Oil Res 27: 232-237. Doi:10.1080/10412905.2015.1007219
Tabata J, Ichiki R T (2017) (1S,3R)-cis-Chrysanthemyl tiglate: sex pheromone of the Striped mealybug, Ferrisia virgata. J Chem Ecol 43:745-752. doi:10.1007/s10886-017-0879-z
Unelius C R, El-Sayed A M, Twidle A, Bunn B, Flores M F, Zaviezo T, Bell V, Bergmann J (2011) Absolute configuration of the citrophilous mealybug sex pheromone. J Chem Ecol 37: 166-172. doi:10.1007/s10886-010-9904-1
Vacas et al., J. Agric. Food Chem. 2019, 67, 9441-9449.
Zou Y, Daane K M, Bentley W J, Millar J G (2010) Synthesis and bioassay of racemic and chiral trans-α-necrodyl isobutyrate, the sex pheromone of the grape mealybug Pseudococcus maritimus. J Agric Food Chem 58: 4977-4982. doi:10.1021/jf904452v

1. A preparation comprising γ-necrodyl isobutyrate and an agriculturally acceptable carrier.

2. The preparation of claim 1, wherein the γ-necrodyl isobutyrate is synthesized from an essential oil of Lavandula luisieri.

3. The preparation of claim 1, wherein said preparation comprises an antioxidant, and/or a preservative.

4. A pest control device comprising γ-necrodyl isobutyrate.

5. A method of controlling a population of Nipaecoccus viridis mealybug population comprising exposing said population to an effective amount of γ-necrodyl isobutyrate, thereby controlling the population of Nipaecoccus viridis.

6. The method of claim 5, wherein said exposing is effected by releasing said γ-necrodyl isobutyrate in a location which is frequented by said Nipaecoccus viridis.

7. The method of claim 6, wherein said location is a field, vineyard or orchard.

8. The method of claim 5, wherein said γ-necrodyl isobutyrate is comprised in a trap.

9. The method of claim 5, wherein said γ-necrodyl isobutyrate is comprised in a sustained release preparation.

10. The method of claim 7, wherein said orchard comprises trees of a species selected from the group consisting of citrus, mango, tamarind and pomegranate.

11. A method of monitoring an amount of Nipaecoccus viridis mealybugs present in a location which is frequented by Nipaecoccus viridis:
(a) setting a trap comprising γ-necrodyl isobutyrate in said location; and
(b) determining the amount of mealybugs in said trap, thereby monitoring the amount of Nipaecoccus viridis mealybugs.

12. The method of claim 11, wherein said determining comprising counting the number of Nipaecoccus viridis mealybugs in said trap.

13. A method of synthesizing γ-necrodol, the method comprising subjecting an α-necrodol to conditions that effect rearrangement of said α-necrodol to thereby generate γ-necrodol.

14. The method of claim 13, wherein said α-necrodol is extracted from an essential oil of Lavandula luisieri.

15. The method of claim 13, wherein said α-necrodol is trans-α-necrodol.

16. A method of synthesizing γ-necrodyl isobutyrate comprising:
(a) synthesizing γ-necrodol according to the method of claim 13; and
(b) reacting said γ-necrodol with isobutyryl chloride, isobutyric acid and/or isobutyric anhydride under conditions that produce γ-necrodyl isobutyrate, thereby synthesizing the γ-necrodyl isobutyrate.

17. The method of claim 16, further comprising purifying said γ-necrodyl isobutyrate following said reacting.

18. A method of synthesizing trans-α-necrodol isobutyrate comprising:
(a) producing trans-α-necrodol from an essential oil of Lavandula luisieri; and
(b) reacting said trans-α-necrodol with isobutyric acid, isobutyryl halide and/or isobutyryl anhydride, under conditions that produce trans-α-necrodol isobutyrate, thereby synthesizing the trans-α-necrodol isobutyrate.

19. A method of synthesizing a mealybug pheromone featuring a necrodane skeleton, the method comprising:
producing a necrodol compound from an essential oil of a plant that comprises at least 10% of said necrodol compound and/or an ester thereof; and
subjecting said necrodol compound to conditions that effect rearrangement and/or esterification of said necrodol, thereby synthesizing the mealybug pheromone.

20. The method of claim 19, wherein said plant is Lavandula luisieri and said necrodol compound is an α-necrodol.

21. The method of claim 20, wherein said mealybug pheromone is an ester of α-necrodol, and wherein the compound is synthesized by subjecting said trans-α-necrodol to conditions that effect esterification of said α-necrodol.

22. The method of claim 19, wherein said mealybug pheromone is an ester of said necrodol, and is synthesized by subjecting said necrodol to conditions that effect esterification of said necrodol.

23. The method of claim 19, wherein said necrodol compound is an α-necrodol and/or a β-necrodol, and wherein the mealybug pheromone is a γ-necrodol or an ester thereof, the method comprising subjecting said α-necrodol and/or a 3-necrodol to conditions that effect rearrangement of said necrodol compound, to thereby obtain said γ-necrodol, and optionally further subjecting said γ-necrodol to conditions that effect esterification, to thereby obtain said ester of γ-necrodol.
